(19) United States
(12) Patent Application Publication
(10) Pub. No.: US 2023/0372274 A1
(43) Pub. Date: Nov. 23, 2023

(54) COMPOSITIONS AND METHODS FOR PERIORBITAL ADMINISTRATION OF EP2 RECEPTOR AGONISTS

(71) Applicant: JENIVISION INC., IRVINE, CA (US)

(72) Inventors: Weizhen WANG, IRVINE, CA (US); DAVID F. WOODWARD, LAKE FOREST, CA (US)

(21) Appl. No.: 18/363,537

(22) Filed: Aug. 1, 2023

Related U.S. Application Data
(63) Continuation of application No. PCT/US22/15339, filed on Feb. 4, 2022.
(60) Provisional application No. 63/146,272, filed on Feb. 5, 2021.

(51) Int. Cl. A61K 31/216; A61K 9/00; A61K 47/14; A61K 9/06; A61P 27/06

(52) U.S. Cl. CPC A61K 31/216; A61K 9/0014; A61K 47/14; A61K 9/06; A61P 27/06

(57) ABSTRACT

Described herein are compositions and methods for the topical administration of prodrugs of EP2 receptor agonists via the periorbital skin. Such compositions and methods are useful for treating various disease and disorders, including glaucoma.

### CROSS REFERENCE

This application is a continuation of International Patent Application No. PCT/US2022/015339, filed on Feb. 4, 2022, which claims the benefit of U. S. Provisional Application No. 63/146,272 filed Feb. 5, 2021, which is incorporated herein by reference in its entirety.

### BACKGROUND OF THE INVENTION

Glaucoma is a leading cause of irreversible blindness worldwide. It has impacted 3.5% of the world's population and it is projected to affect approximately 111 million people globally by 2040. Current treatments of glaucoma suffer from many side effects, including irritation to the eye and surrounding tissues due to irritants in the formulations, which in some cases become so severe that the patient must discontinue treatment. There exists a need for improved treatments of glaucoma.

### SUMMARY OF THE INVENTION

One embodiment provides a method of lowering intraocular pressure in a patient suffering from elevated intraocular pressure comprising administering a therapeutically effective amount of a composition comprising 3-[(3′-fluoro-4-fluorobiphenyl-3-carbonyl) amino] phenoxyacetic acid isopropyl ester to the periorbital skin of an eye of the patient wherein the 3-[(3′-fluoro-4-fluorobiphenyl-3-carbonyl) amino] phenoxyacetic acid isopropyl ester is administered as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

One embodiment provides a method of treating glaucoma in a patient suffering from glaucoma comprising administering a therapeutically effective amount of a composition comprising 3-[(3′-fluoro-4-fluorobiphenyl-3-carbonyl) amino] phenoxyacetic acid isopropyl ester to the periorbital skin of an eye of the patient wherein the 3-[(3′-fluoro-4-fluorobiphenyl-3-carbonyl) amino] phenoxyacetic acid isopropyl ester is administered as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

One embodiment provides a method of lowering intraocular pressure in a patient suffering from glaucoma comprising administering a therapeutically effective amount of a composition comprising 3-[(3′-fluoro-4-fluorobiphenyl-3-carbonyl)amino]phenoxyacetic acid isopropyl ester to the periorbital skin of an eye of the patient wherein the 3-[(3′-fluoro-4-fluorobiphenyl-3-carbonyl)amino] phenoxyacetic acid isopropyl ester is administered as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

Another embodiment provides the method, wherein the composition comprising 3-[(3′-fluoro-4-fluorobiphenyl-3-carbonyl) amino] phenoxyacetic acid isopropyl ester is administered to the periorbital skin of each eye of the patient twice per day, once per day, once every other day, once every three days, once every four days, or once every seven days. Another embodiment provides the method, wherein the topical composition is administered once per day.

Another embodiment provides the method, wherein the composition is in the form of an ointment. Another embodiment provides the method, wherein the ointment comprises petrolatum. Another embodiment provides the method, wherein the ointment comprises petrolatum and medium-chain triglycerides. Another embodiment provides the method, wherein the medium-chain triglycerides comprise a mixture of C6, C8, C10 and C12 fatty acids. Another embodiment provides the method, wherein the medium-chain triglycerides comprise a mixture of caprylic acid and capric acid.

Another embodiment provides the method, wherein the ointment comprises petrolatum and medium-chain triglyceride in the ratio of about 1:1 (v/v), about 2:1 (v/v), about 3:1 (v/v), about 4:1 (v/v), about 5:1 (v/v), or about 6:1 (v/v). Another embodiment provides the method, wherein the ointment comprises petrolatum and medium-chain triglyceride in the ratio of about 4:1 (v/v).

Another embodiment provides the method, wherein the composition is administered from a device which releases a preselected dosage in a uniform manner onto the periorbital skin of the patient. Another embodiment provides the method, wherein the method comprises administering the composition to the periorbital skin above the upper eyelid, below the lower eyelid, or both above the upper and below the lower eyelids. Another embodiment provides the method, wherein the method comprises administering the composition to the periorbital skin above the upper eyelid. Another embodiment provides the method, wherein the method comprises administering the composition to the periorbital skin below the lower eyelid. Another embodiment provides the method, wherein the method comprises administering the composition to the periorbital skin both above the upper and below the lower eyelids.

Another embodiment provides the method, wherein the composition is administered as a topical composition to deliver from about 0.0005 micrograms per eye of 3-[(3′-fluoro-4-fluorobiphenyl-3-carbonyl) amino] phenoxyacetic acid isopropyl ester per administration to about 0.005 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver from about 0.005 micrograms per eye of 3-[(3′-fluoro-4-fluorobiphenyl-3-carbonyl) amino] phenoxyacetic acid isopropyl ester per administration to about 0.05 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver from about 0.05 micrograms per eye of 3-[(3′-fluoro-4-fluorobiphenyl-3-carbonyl) amino] phenoxyacetic acid isopropyl ester per administration to about 0.5 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver from about 0.5 micrograms per eye of 3-[(3′- fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver about 0.3 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver ab out 0.6 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver about 1.0 micrograms per eye of 3-[(3′-fluoro-4-fluorobiphenyl-3-carbonyl) amino] phenoxyacetic acid isopropyl ester per administration.

One embodiment provides a pharmaceutical composition suitable for topical periorbital administration, comprising 3-[(3′-fluoro-4-fluorobiphenyl-3-carbonyl) amino] phenoxyacetic acid isopropyl ester and a pharmaceutically acceptable excipient, wherein the pharmaceutical composition is configured to dispense from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

Another embodiment provides the pharmaceutical composition, wherein the composition is in the form of an ointment. Another embodiment provides the pharmaceutical composition, wherein the ointment comprises petrolatum. Another embodiment provides the pharmaceutical composition, wherein the ointment comprises petrolatum and medium-chain triglycerides. Another embodiment provides the pharmaceutical composition, wherein the medium-chain triglyceride comprises a C6, a C8, a C10, or a C12 fatty acid, or any combination thereof. Another embodiment provides the pharmaceutical composition, wherein the medium-chain triglycerides comprise a mixture of caprylic acid and capric acid.

Another embodiment provides the pharmaceutical composition, wherein the ointment comprises petrolatum and medium chain triglycerides in the ratio of about 1:1 (v/v), about 2:1 (v/v), about 3:1 (v/v), about 4:1 (v/v), about 5:1 (v/v), or about 6:1 (v/v). Another embodiment provides the pharmaceutical composition, wherein the ointment comprises petrolatum and medium chain triglycerides in the ratio of about 4:1 (v/v).

Another embodiment provides the pharmaceutical composition, wherein the composition is administered from a device which releases a preselected dosage in a uniform manner onto the periorbital skin of the patient.

Another embodiment provides the pharmaceutical composition, wherein the composition is configured to deliver from about 0.0005 micrograms per eye of 3-[(3′-fluoro-4-fluorobiphenyl-3-carbonyl) amino] phenoxyacetic acid isopropyl ester per administration to about 0.005 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver from about 0.005 micrograms per eye of 3-[(3′-fluoro-4-fluorobiphenyl-3-carbonyl) amino] phenoxyacetic acid isopropyl ester per administration to about 0.05 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver from about 0.05 micrograms per eye of 3-[(3′-fluoro-4-fluorobiphenyl-3-carbonyl) amino] phenoxyacetic acid isopropyl ester per administration to about 0.5 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver from about 0.5 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 0.3 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 0.6 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 1.0 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 2.0 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 3.0 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 4.0 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 5.0 micrograms per eye of 3-[(3′-fluoro-4-fluorobiphenyl-3-carbonyl) amino] phenoxyacetic acid isopropyl ester per administration.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

FIG. 1 shows a representation of an eye with normal vision and an eye with glaucoma.

FIG. 2 compares the effect of 9 micrograms bimatoprost (30 pi of 0.03% Lumigan® ophthalmic solution) topical ocular surface administration once daily per eye for 4 days with single dose periorbital skin administration of [(3′-fluoro-4-fluorobiphenyl-3-carbonyl) amino] phenoxyacetic acid isopropyl ester ointment at 10 micrograms, 20 micrograms or 30 micrograms [(3′-fluoro-4-fluorobiphenyl-3-carbonyl) amino] phenoxyacetic acid isopropyl ester on intraocular pressure (TOP) in ocular normotensive Cynomolgus monkeys (Mean±SEM, n=4/group).

FIG. 3 shows the effect of JV-GL1 Ointment 0.2 micrograms once daily per eye on IOP in ocular normotensive Cynomolgus monkeys (OD, Mean±SEM, n=3 of Group 1 on Days 15-21). Animals were unilaterally treated via periorbital skin administration of [(3′-fluoro-4-fluorobiphenyl-3-carbonyl) amino] phenoxyacetic acid isopropyl ester ointment once daily on Days 15-21. The right eye of the control group (Group 2) received ointment vehicle once daily on Days 8-14 and no treatment on Days 15-21. OD=right eye.

FIG. 4 shows the anatomy of the periorbital region of the eye.

FIG. 5 shows the effect on IOP of human subjects after administering different concentrations of JV-GL1 ointment once daily in both eyes.

### DETAILED DESCRIPTION OF THE INVENTION

Described herein are methods and compositions useful for treating glaucoma and/or alleviating elevated intraocular pressure. In some embodiments, said methods and compositions utilize [(3′-fluoro-4-fluorobiphenyl-3-carbonyl) amino] phenoxyacetic acid isopropyl ester (a.k.a. JV-GL1) administered as a topical formulation to the periorbital skin of a subject.

Surprisingly, it has been shown herein that periorbital administration of a low dose (e.g. 5 micrograms per eye or less) of [(3′-fluoro-4-fluorobiphenyl-3-carbonyl) amino] phenoxyacetic acid isopropyl ester to the periorbital skin of a patient is effective to alleviate intraocular pressure associated with glaucoma. Periorbital application of [(3′-fluoro-4-fluorobiphenyl-3-carbonyl) amino] phenoxyacetic acid isopropyl ester at low dose also eliminated ocular hyperemia observed when [(3′-fluoro-4-fluorobiphenyl-3-carbonyl) amino] phenoxyacetic acid isopropyl ester was administered as topical ophthalmic eye drops.

Additionally, the low doses described herein eliminate many of the side effects associated with higher doses of [(3′-fluoro-4-fluorobiphenyl-3-carbonyl) amino] phenoxyacetic acid isopropyl ester and other treatments of glaucoma, including periorbital skin swelling and hyperemia of the periorbital skin and ocular surface. The mitigation of these side effects thus enables a patient to continue a daily or other frequent dosage regimen of [(3′-fluoro-4-fluorobiphenyl-3-carbonyl) amino] phenoxyacetic acid isopropyl ester without any need to discontinue treatment. The ability to repeatedly administer, such as, for example once or twice daily, low doses of the compound without adverse effects thus eliminates an oscillation of intraocular pressure (the so-called “yo-yo effect”) in patients who stop and re-start treatment or administer less frequently, which can reduce the efficacy of the therapy.

One embodiment provides a method of lowering intraocular pressure in a patient suffering from elevated intraocular pressure comprising administering a therapeutically effective amount of a composition comprising 3-[(3′-fluoro-4-fluorobiphenyl-3-carbonyl) amino] phenoxyacetic acid isopropyl ester to the periorbital skin of an eye of the patient wherein the 3-[(3′-fluoro-4-fluorobiphenyl-3-carbonyl) amino] phenoxyacetic acid isopropyl ester is administered as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

One embodiment provides a method of treating glaucoma in a patient suffering from glaucoma comprising administering a therapeutically effective amount of a composition comprising 3-[(3′-fluoro-4-fluorobiphenyl-3-carbonyl) amino] phenoxyacetic acid isopropyl ester to the periorbital skin of an eye of the patient wherein the 3-[(3′-fluoro-4-fluorobiphenyl-3-carbonyl) amino] phenoxyacetic acid isopropyl ester is administered as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

One embodiment provides a method of lowering intraocular pressure in a patient suffering from glaucoma comprising administering a therapeutically effective amount of a composition comprising 3-[(3′-fluoro-4-fluorobiphenyl-3-carbonyl)amino]phenoxyacetic acid isopropyl ester to the periorbital skin of an eye of the patient wherein the 3-[(3′-fluoro-4-fluorobiphenyl-3-carbonyl)amino] phenoxyacetic acid isopropyl ester is administered as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

Another embodiment provides the method, wherein the composition comprising 3-[(3′- fluoro-4-fluorobiphenyl-3-carbonyl) amino] phenoxyacetic acid isopropyl ester is administered to the periorbital skin of each eye of the patient twice per day, once per day, once every other day, once every three days, once every four days, or once every seven days. Another embodiment provides the method, wherein the topical composition is administered once per day.

Another embodiment provides the method, wherein the composition is in the form of an ointment. Another embodiment provides the method, wherein the ointment comprises petrolatum.

Another embodiment provides the method, wherein the ointment comprises petrolatum and medium-chain triglycerides. Another embodiment provides the method, wherein the medium-chain triglycerides comprise a mixture of C6, C8, C10 and C12 fatty acids. Another embodiment provides the method, wherein the medium-chain triglycerides comprise a mixture of caprylic acid and capric acid.

Another embodiment provides the method, wherein the ointment comprises petrolatum and medium-chain triglyceride in the ratio of about 1:1 (v/v), about 2:1 (v/v), about 3:1 (v/v), about 4:1 (v/v), about 5:1 (v/v), or about 6:1 (v/v). Another embodiment provides the method, wherein the ointment comprises petrolatum and medium-chain triglyceride in the ratio of about 4:1 (v/v).

Another embodiment provides the method, wherein the composition is administered from a device which releases a preselected dosage in a uniform manner onto the periorbital skin of the patient. Another embodiment provides the method, wherein the method comprises administering the composition to the periorbital skin above the upper eyelid, below the lower eyelid, or both above the upper and below the lower eyelids. Another embodiment provides the method, wherein the method comprises administering the composition to the periorbital skin above the upper eyelid. Another embodiment provides the method, wherein the method comprises administering the composition to the periorbital skin below the lower eyelid. Another embodiment provides the method, wherein the method comprises administering the composition to the periorbital skin both above the upper and below the lower eyelids.

Another embodiment provides the method, wherein the composition is administered as a topical composition to deliver from about 0.0005 micrograms per eye of 3-[(3′-fluoro-4-fluorobiphenyl-3-carbonyl) amino] phenoxyacetic acid isopropyl ester per administration to about 0.005 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver from about 0.005 micrograms per eye of 3-[(3′-fluoro-4-fluorobiphenyl-3-carbonyl) amino] phenoxyacetic acid isopropyl ester per administration to about 0.05 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver from about 0.05 micrograms per eye of 3-[(3′-fluoro-4-fluorobiphenyl-3-carbonyl) amino] phenoxyacetic acid isopropyl ester per administration to about 0.5 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver from about 0.5 micrograms per eye of 3-[(3-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver about 0.3 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver about 0.6 micrograms per eye of 3-[(3′-fluoro-4-fluorobiphenyl-3-carbonyl) amino] phenoxyacetic acid isopropyl ester per administration. Another embodiment provides the method, wherein the composition is administered as a topical composition to deliver about 1.0 micrograms per eye of 3-[(3′-fluoro-4-fluorobiphenyl-3-carbonyl) amino] phenoxyacetic acid isopropyl ester per administration.

One embodiment provides a pharmaceutical composition suitable for topical periorbital administration, comprising 3-[(3′-fluoro-4-fluorobiphenyl-3-carbonyl) amino] phenoxyacetic acid isopropyl ester and a pharmaceutically acceptable excipient, wherein the pharmaceutical composition is configured to dispense from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

Another embodiment provides the pharmaceutical composition, wherein the composition is in the form of an ointment. Another embodiment provides the pharmaceutical composition, wherein the ointment comprises petrolatum. Another embodiment provides the pharmaceutical composition, wherein the ointment comprises petrolatum and medium-chain triglycerides. Another embodiment provides the pharmaceutical composition, wherein the medium-chain triglyceride comprises a C6, a C8, a C10, or a C12 fatty acid, or any combination thereof. Another embodiment provides the pharmaceutical composition, wherein the medium-chain triglycerides comprise a mixture of caprylic acid and capric acid.

Another embodiment provides the pharmaceutical composition, wherein the ointment comprises petrolatum and medium chain triglycerides in the ratio of about 1:1 (v/v), about 2:1 (v/v), about 3:1 (v/v), about 4:1 (v/v), about 5:1 (v/v), or about 6:1 (v/v). Another embodiment provides the pharmaceutical composition, wherein the ointment comprises petrolatum and medium chain triglycerides in the ratio of about 4:1 (v/v).

Another embodiment provides the pharmaceutical composition, wherein the composition is administered from a device which releases a preselected dosage in a uniform manner onto the periorbital skin of the patient.

Another embodiment provides the pharmaceutical composition, wherein the composition is configured to deliver from about 0.0005 micrograms per eye of 3-[(3′-fluoro-4-fluorobiphenyl-3-carbonyl) amino] phenoxyacetic acid isopropyl ester per administration to about 0.005 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver from about 0.005 micrograms per eye of 3-[(3′-fluoro-4-fluorobiphenyl-3-carbonyl) amino] phenoxyacetic acid isopropyl ester per administration to about 0.05 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver from about 0.05 micrograms per eye of 3-[(3′-fluoro-4-fluorobiphenyl-3-carbonyl) amino] phenoxyacetic acid isopropyl ester per administration to about 0.5 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver from about 0.5 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 0.3 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 0.6 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 1.0 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 2.0 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 3.0 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 4.0 micrograms per eye of 3-[(3′-fluoro-4-fluorobiphenyl-3-carbonyl) amino] phenoxyacetic acid isopropyl ester per administration. Another embodiment provides the pharmaceutical composition, wherein the composition is configured to deliver about 5.0 micrograms per eye of 3-[(3′-fluoro-4-fluorobiphenyl-3-carbonyl) amino] phenoxyacetic acid isopropyl ester per administration.

### Glaucoma

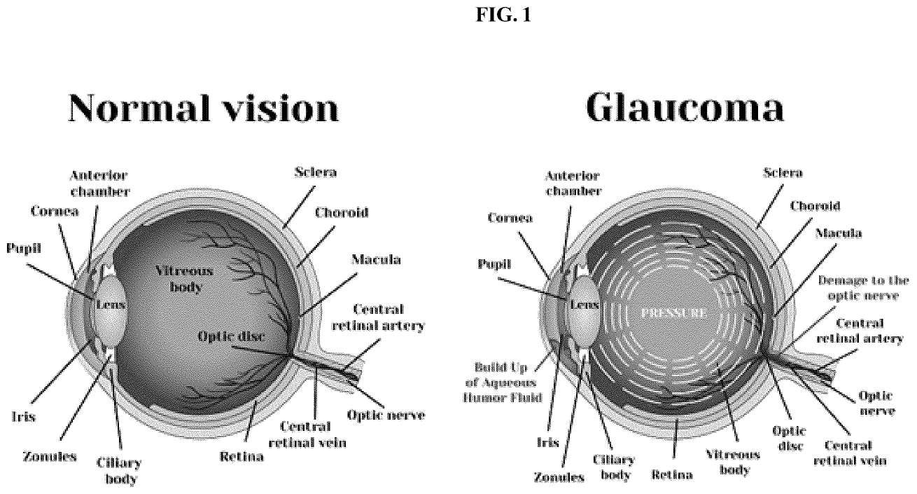

Glaucoma is a heterogeneous group of chronic and progressive ocular neuropathies that can result in progressive injury to the optic nerve, with retinal ganglion cell death and adverse effects on the visual field (Cvenkel B and Kolko M. Current medical therapy and future trends in the management of glaucoma treatment. J Ophthalmology, 2020; Article ID 6138132, https://doi.org/10.1155/2020/6138132). Comparison of a normal human eye versus one with glaucoma is shown in FIG. 1.

Glaucoma is a leading cause of irreversible blindness worldwide. It has impacted 3.5% of the world's population and it is projected to affect approximately 111 million people globally by 2040 (Thompson AC, Jammal AA and Medeiros FA. A review of deep learning for screening, diagnosis, and detection of glaucoma progression. Trans Vis Sci Tech, 2020; 9(2):42, https://doi.org/10.1167/tvst.9.2.42; Trivli A, Zervou M, Goulielmos GN et al. Primary open angle glaucoma genetics: The common variants and their clinical associations (review). Molecular Medicine Reports, 2020; 22:1130-1110). The National Eye Institute (National Eye Institute. Glaucoma Data and Statistics. National Institutes of Health. Updated July 19, 2019. https://www.nei.nih.gov/learn-ab out-eye-health/resources-for-health-educators/eye-health-data-and-statistics/glaucoma-data-and-statistics) reported that in 2010 glaucoma affected 1.9% of individuals in the US aged 40 and above and in 2030 it is projected to affect 4% of the US population. Doucette and Walter (Doucette LP and Walter MA. Prostaglandins in the eye: Function, expression, and roles in glaucoma. Ophthalmic Genetics 2017; 38:2: 108 -116. doi: 10.3109/13816810.2016.1164193) projected that in 2020 approximately 80 million people worldwide would be affected by glaucoma.

The exact etiology of glaucoma remains largely unknown, but the primary modifiable risk factor for this disease is intraocular pressure (IOP). The balance between secretion of aqueous humor by the ciliary epithelium and its drainage through two independent pathways, the trabecular meshwork (conventional) and uveoscleral (unconventional) outflow pathways, determines IOP. The uveoscleral pathway is reported to account for 5 to 15% of IOP regulation, with the conventional pathway predominating and accounting for the remainder (Angeli A and Supuran CT. Prostaglandin receptor agonists as antiglaucoma agents (a patent review 2013 — 2018). Expert Opinion on Therapeutic Patents, 2019; 29(10):793 -803. doi: 10.1080/13543776.2019.1661992; Doucette and Walter, 2017). In patients with open-angle glaucoma, there is increased resistance to the aqueous outflow through the conventional outflow pathway (Ascott TS, Vranka JA, Keller KE et al. Normal and glaucomatous outflow regulation. Progress in Retinal and Eye Research, 2020. https://doi.org/10.1016/j.preteyeres.2020.100897). Because glaucoma usually progresses without causing symptoms, the disease is generally advanced, with substantial amounts of neural damage when it is diagnosed (Angeli and Supuran, 2019; Dietze J, Blair K and Havens S J. Glaucoma. 2020 Jun 30. In: StatPearls [Internet]. Treasure Island (FL): StatPearls Publishing; 2020 January-. PMID: 30855805). The result is vision loss and a concomitant reduction in quality of life and the ability to perform daily activities.

The only proven and generally accepted treatment to reduce the risk of further progression of glaucomatous optic neuropathy continues to be lowered IOP (Ascott et al., 2020; Cvenkel and Kolko 2020; Garway-Heath DF, Crabb DP, Bunce C et al. Latanoprost for open-angle glaucoma (UKGTS): A randomized, multicentre, placebo-controlled trial. Lancet, 2015; 385:1295-1304; Heijl A, Leske M C, Bengtsson B et al. Reduction of intraocular pressure and glaucoma progression: results from the Early Manifest Glaucoma Trial. Arch Ophthalmol, 2002 ;120:1268-1279; Kass M A, Heuer D K, Higginbotham E J et al. The Ocular Hypertension Treatment Study: A randomized trial determines that topical ocular hypotensive medication delays or prevents the onset of primary open-angle glaucoma. Arch Ophthalmol, 2002; 120:701-713; Trivli et al. 2020). There are four main classes of approved drugs used to treat glaucoma in the US. Prostaglandin F_{2α} analogs (e.g. bimatoprost, latanoprost, travoprost, tafluprost) are the first-line of medical treatment for glaucoma (Doucette and Walter, 2017). Other drug classes include β adrenergic blockers (e.g., timolol, betaxolol), carbonic anhydrase inhibitors (e.g., dorzolamide, brinzolamide), and α₋₂ adrenergic agonists (e.g., brimonidine). The newest FDA approvals occurred in 2017 for latanoprostene bunod (a prostaglandin F_{2α} analog) and netarsudil (a Rho kinase inhibitor), and in 2019, the fixed combination netarsudil/latanoprost was launched. Prostaglandin analogs improve uveoscleral and pressure dependent outflow whereas β adrenergic blockers and carbonic anhydrase inhibitors decrease aqueous humor production and α₋₂ adrenergic agonists act by suppressing aqueous humor secretion and increasing uveoscleral outflow (Cvenkel and Kolko 2020; Wang H, Deng Y, Wan L and Huang L. A comprehensive map of disease networks and molecular drug discoveries for glaucoma. Scientific Reports, 2020; 10:9719. https://doi.org/10.1038/s41598-020-66350-w). There are limitations associated with the traditional glaucoma treatments as they are often associated with adverse systemic effects (Ascot et al., 2020). Some surgical interventions exist for treating this disease. Therefore, there continues to be a medical need for improved, alternative treatment options for glaucoma.

[(3′-Fluoro-4-fluorobiphenyl-3-carbonyl) amino] Phenoxyacetic Acid Isopropyl Ester

The compound 3-[(3′-fluoro-4-fluorobiphenyl-3-carbonyl) amino] phenoxyacetic acid isopropyl ester (also known as JV-GL1) is a prodrug of an EP2 receptor agonist (Coleman RA, Woodrooffe AJ, Woodward DF et al. The affinity, intrinsic activity and selectivity of a structurally novel EP2 receptor agonist at human prostanoid receptors. Br J Pharmacol, 2019; 176:687-98). It exhibits high potency and an extended duration of action (Woodward D F, Wang J W, Coleman R A, et al. A highly effective and ultra-long acting anti-glaucoma drug with a new periorbital delivery method. J Ocular Pharmacol Ther, 2019; 35: 265-277). Furthermore, 3-[(3′- fluoro-4-fluorobiphenyl-3-carbonyl) amino] phenoxyacetic acid isopropyl ester is not administered directly into the eye via eye drops like other glaucoma drugs, but is applied topically to the periorbital skin, a route that is intended to minimize local ocular irritation (see, U.S. Pat. No. 9,820,954). Finally, unlike current pharmaceutical glaucoma products which have associated adverse systemic effects, 3-[(3′-fluoro-4-fluorobiphenyl-3-carbonyl) amino] phenoxyacetic acid isopropyl ester is not anticipated to produce systemic effects due to the low doses required to produce ophthalmic efficacy and the anticipated very low circulating levels of the pro-drug and active moiety. Because of these factors, 3-[(3′-fluoro-4-fluorobiphenyl-3-carbonyl) amino] phenoxyacetic acid isopropyl ester, when administered to the periorbital region, represents a potential advancement in treatment options for glaucoma.

The chemical structure of 3-[(3′-fluoro-4-fluorobiphenyl-3-carbonyl) amino] phenoxyacetic acid isopropyl ester is shown below.

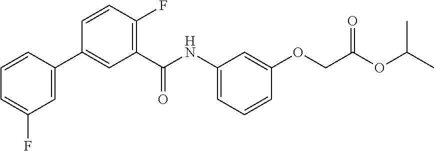 [SMILES: CC(C)OC(=O)COc1cccc(NC(=O)c2cc(-c3cccc(F)c3)ccc2F)c1]

In situ, JV-GL1 is converted to its active form, 3-[(3′-fluoro-4-fluorobiphenyl-3-carbonyl) amino] phenoxyacetic acid, following enzymatic hydrolysis in ocular tissues. The structure of which is shown below.

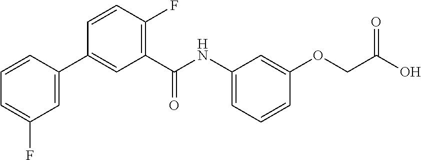 [SMILES: O=C(O)COc1cccc(NC(=O)c2cc(-c3cccc(F)c3)ccc2F)c1]

The acid form, 3-[(3′-fluoro-4-fluorobiphenyl-3-carbonyl) amino] phenoxyacetic acid, was shown to be a selective and high affinity (pKi 8.5) ligand at human recombinant EP₂ receptors (Coleman et al., 2019). In addition to high affinity binding, it was a potent and full EP₂ receptor agonist with a high level of selectivity and negligible affinity for EP₁, EP₃, EP₄, DP, FP, IP and TP receptors (Woodward et al., 2019). In HEK cells over-expressing human recombinant EP₂ receptors, JV-GL1 free acid displayed a potency of pEC₅₀9.9 producing a maximal elevation of cAMP levels comparable to that of the endogenous agonist PGE2.

### Formulations

Certain aspects of the present disclosure relates to a pharmaceutical composition comprising a pharmaceutically acceptable carrier. In some embodiments, the pharmaceutically acceptable carrier is a carrier sufficient for topical and/or transdermal administration/application.

In some embodiments, the pharmaceutically acceptable carrier is suitable for topical or transdermal applications/administrations. Examples of carriers suitable for use in a topical or transdermal application/administration may include, but are not limited to, ointments, pastes, creams, suspensions, emulsions, fatty ointments, gels, powders, lotions, solutions, sprays, patches, microneedle arrays, and inhalants. In some embodiments, the pharmaceutically acceptable carrier comprises one or more of an ointment, paste, cream, suspension, emulsion, fatty ointment, gel, powder, lotion, solution, spray, and an inhalant. In some embodiments, the pharmaceutically acceptable carrier comprises an ointment. In some embodiments, the pharmaceutically acceptable carrier comprises a paste. In some embodiments, the pharmaceutically acceptable carrier comprises a cream. In some embodiments, the pharmaceutically acceptable carrier comprises a suspension. In some embodiments, the pharmaceutically acceptable carrier comprises an emulsion. In some embodiments, the pharmaceutically acceptable carrier comprises a gel. In some embodiments, the pharmaceutically acceptable carrier comprises a powder. In some embodiments, the pharmaceutically acceptable carrier comprises a lotion. In some embodiments, the pharmaceutically acceptable carrier comprises a solution. In some embodiments, the pharmaceutically acceptable carrier comprises a spray. In some embodiments, the pharmaceutically acceptable carrier comprises an inhalant. In some embodiments, the pharmaceutical carrier comprises a patch (e.g. a patch that adheres to the skin).

In some embodiments, the pharmaceutically acceptable carrier comprises a combination of two, three, four, five or more different pharmaceutically acceptable carriers suitable for topical or transdermal applications/administrations.

In some embodiments, the pharmaceutically acceptable carrier further comprises one or more additional components. Examples of additional components may include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); wetting agents (e.g., sodium lauryl sulphate, etc.); salt solutions; alcohols; polyethylene glycols; gelatin; lactose; amylase; magnesium stearate; talc; silicic acid; viscous paraffin; hydroxymethylcellulose; polyvinylpyrrolidone; sweetenings; flavorings; perfuming agents; colorants; moisturizers; sunscreens; antibacterial agents; agents able to stabilize polynucleotides or prevent their degradation, and the like.

Provided herein in some embodiments are compositions comprising 3-[(3′-fluoro-4-fluorobiphenyl-3-carbonyl) amino] phenoxyacetic acid isopropyl ester suitable for application to the periorbital skin region of the eye of a subject. In some embodiments, the composition is in the form of an ointment, a cream, or a lotion.

In some embodiments, the composition is in the form of an ointment. In some embodiments, the ointment comprises a semi-solid oleaginous base material. In some embodiments, the ointment comprises a petroleum base, a mineral oil, a polyol, a triglyceride, or any combination thereof. In some embodiments, the ointment comprises a petroleum base. In some embodiments, the ointment comprises petrolatum. In some embodiments, the ointment comprises petrolatum, a triglyceride, or any combination thereof. In some emb odiments, the ointment comprises petrolatum and a triglyceride.

In some embodiments, the ointment comprises a triglyceride. In some embodiments, the triglyceride is a medium-chain or a long-chain triglyceride. In some embodiments, the triglyceride is a medium-chain triglyceride. In some embodiments, the medium-chain triglyceride comprises 2 or 3 medium length fatty acids. In some embodiments, the medium-chain triglyceride comprises C6 or larger fatty acids. In some emb odiments, the medium chain triglyceride comprises C6 to C12 fatty acids. In some embodiments, the medium-chain triglyceride comprises a mixture of C6 to C12 fatty acids. In some embodiments, the medium-chain triglyceride comprises fatty acids selected from C6, C8, C10, and C12 fatty acids, or a mixture thereof. In some embodiments, the medium-chain triglyceride comprises caproic acid, caprylic acid, capric acid, lauric acid, or any combination thereof. In some embodiments, the medium-chain triglyceride comprises caprylic acid, capric acid, or a combination thereof. In some embodiments, the medium-chain triglyceride comprises caprylic acid and capric acid. In some embodiments, the medium-chain triglyceride comprises caprylic acid and capric acid in a ratio of about 4:1 (w/w), about 4:3 (w/w), about 3:1 (w/w), about 3:2 (w/w), about 1:1 (w/w), about 2:3 (w/w), about 1:3 (w/w), about 3:4 (w/w), or about 1:4 (w/w). In some embodiments, the ratio is from about 1:1 (w/w) to about 4:1 (w/w). In some embodiments, the ratio is about 3:2 (w/w). In some embodiments, the medium-chain triglyceride comprises at least 75%, at least 80%, at least 85%, at least 90%, at least 91%, at least 92%, at least 93%, at least 94%, or at least 95% C6 to C12 fatty acids as compared to other fatty acids (w/w).

In some embodiments, the medium-chain triglyceride is derived from a natural source. In some embodiments, the medium-chain triglyceride is derived from coconut, palm, or palm kernel, or combinations thereof. In some embodiments, the medium-chain triglyceride is derived from coconut, or palm. In some embodiments, the medium-chain triglyceride is the oil extracted from the endosperm of coconut or palm. In some embodiments, the medium-chain triglyceride is National Food (NF) grade (NF) or US Pharmacopeia (USP) grade.

In some embodiments, the ointment comprises a mixture of petrolatum and a medium-chain triglyceride. In some embodiments, the ratio of petrolatum to medium-chain triglyceride is from about 10:1 (v/v) to about 1:2 (v/v). In some embodiments, the ratio of petrolatum to medium-chain triglyceride is from about 6:1 (v/v) to about 1:1 (v/v). In some embodiments, the ratio of petrolatum to medium-chain triglyceride is from about 6:1 (v/v) to about 1:1 (v/v), from about 5:1 (v/v) to about 1:1 (v/v), from about 4:1 (v/v) to about 1:1 (v/v), from about 3:1 (v/v) to about 2:1 (v/v), or from about 3:2 (v/v) to about 1:1 (v/v). In some embodiments, the ratio of petrolatum to medium-chain triglyceride is about 1:1 (v/v), about 2:1 (v/v), about 3:1 (v/v), about 4:1 (v/v), about 5:1 (v/v), or about 6:1 (v/v). In some embodiments, the ratio of petrolatum to medium-chain triglyceride is about 1:1 (v/v). In some embodiments, the ratio of petrolatum to medium-chain triglyceride is about 2:1 (v/v). In some embodiments, the ratio of petrolatum to medium-chain triglyceride is about 4:1 (v/v).

In some embodiments, the composition comprises 3-[(3′-fluoro-4-fluorobiphenyl-3-carbonyl) amino] phenoxyacetic acid isopropyl ester in an amount of up to 0.01% (w/w), up to 0.009% (w/w), up to 0.008% (w/w), up to 0.007% (w/w) up to 0.006% (w/w), or up to 0.005% (w/w). In some embodiments, the composition comprises 3-[(3′-fluoro-4-fluorobiphenyl-3-carbonyl) amino] phenoxyacetic acid isopropyl ester in an amount of from about 0.000001% (w/w) to 0.01% (w/w). In some embodiments, the composition comprises 3-[(3′-fluoro-4-fluorobiphenyl-3-carbonyl) amino] phenoxyacetic acid isopropyl ester in an amount of from about 0.000001% (w/w) to 0.008% (w/w). In some embodiments, the composition comprises 3-[(3′-fluoro-4-fluorobiphenyl-3-carbonyl) amino] phenoxyacetic acid isopropyl ester in an amount of from about 0.000001% (w/w) to 0.007% (w/w). In some embodiments, the composition comprises 3-[(3′-fluoro-4-fluorobiphenyl-3-carbonyl) amino] phenoxyacetic acid isopropyl ester in an amount of from about 0.000001% (w/w) to 0.006% (w/w). In some embodiments, the composition comprises 3-[(3′-fluoro-4-fluorobiphenyl-3-carbonyl) amino] phenoxyacetic acid isopropyl ester in an amount of from about 0.000001% (w/w) to about 0.005% (w/w). In some embodiments, the composition comprises 3-[(3′-fluoro-4-fluorobiphenyl-3-carbonyl) amino] phenoxyacetic acid isopropyl ester in an amount of from about 0.00001% (w/w) to 0.01% (w/w). In some embodiments, the composition comprises 3-[(3′-fluoro-4-fluorobiphenyl-3-carbonyl) amino] phenoxyacetic acid isopropyl ester in an amount of from about 0.00001% (w/w) to about 0.005% (w/w). In some embodiments, the composition comprises 3-[(3′-fluoro-4-fluorobiphenyl-3-carbonyl) amino] phenoxyacetic acid isopropyl ester in an amount of from about 0.0001% (w/w) to 0.01% (w/w). In some embodiments, the composition comprises 3-[(3′-fluoro-4-fluorobiphenyl-3-carbonyl) amino] phenoxyacetic acid isopropyl ester in an amount of from about 0.0001% (w/w) to about 0.005% (w/w). In some embodiments, the composition comprises 3-[(3′-fluoro-4-fluorobiphenyl-3-carbonyl) amino] phenoxyacetic acid isopropyl ester in an amount of from about 0.001% (w/w) to 0.01% (w/w). In some embodiments, the composition comprises 3-[(3′-fluoro-4-fluorobiphenyl-3-carbonyl) amino] phenoxyacetic acid isopropyl ester in an amount of from about 0.001% (w/w) to about 0.005% (w/w). In some embodiments, the composition comprises 3-[(3′-fluoro-4-fluorobiphenyl-3-carbonyl) amino] phenoxyacetic acid isopropyl ester in an amount of from about 0.000001% (w/w) to about 0.009% (w/w), 0.00001% (w/w) to about 0.009% (w/w), 0.001% (w/w) to about 0.009% (w/w), 0.001% (w/w) to about 0.008% (w/w), 0.0001% (w/w) to about 0.007% (w/w), 0.0001% (w/w) to about 0.006% (w/w), 0.0001% (w/w) to about 0.005% (w/w), or 0.001% (w/w) to about 0.005% (w/w). In some embodiments, the composition comprises 3-[(3′-fluoro-4-fluorobiphenyl-3-carbonyl) amino] phenoxyacetic acid isopropyl ester in an amount of about 0.001% (w/w), about 0.002% (w/w), about 0.003% (w/w), about 0.004% (w/w), about 0.005% (w/w), about 0.006% (w/w), about 0.007% (w/w), about 0.008% (w/w), or about 0.009% (w/w). In some embodiments, the composition comprises 3-[(3′-fluoro-4-fluorobiphenyl-3-carbonyl) amino] phenoxyacetic acid isopropyl ester in an amount of about 0.0001% (w/w), about 0.0002% (w/w), about 0.0003% (w/w), about 0.0004% (w/w), about 0.0005% (w/w), about 0.0006% (w/w), about 0.0007% (w/w), about 0.0008% (w/w), or about 0.0009% (w/w).

In some embodiments, the composition comprises 3-[(3 ‘-fluoro-4-fluorobiphenyl-3-carbonyl) amino] phenoxyacetic acid isopropyl ester in an amount from about 0.01% to about 0.025% (w/w). In some embodiments, the composition comprises 3-[(3’-fluoro-4-fluorobiphenyl-3-carbonyl) amino] phenoxyacetic acid isopropyl ester in an amount from about 0.01% to about 0.05% (w/w).

In some embodiments, the composition is free from preservatives. In some embodiments, the composition is free from benzalkonium chloride. In some embodiments, the composition is free from water.

### Methods of Treatment

Provided herein in some embodiments is a method of lowering intraocular pressure in a patient suffering from elevated intraocular pressure. In some embodiments is a method of lowering intraocular pressure in a patient suffering from glaucoma. Also provided herein in some embodiments is a method of treating glaucoma in a patient suffering from glaucoma.

Provided herein is a method of lowering intraocular pressure in a patient suffering from elevated intraocular pressure comprising administering to the periorbital skin of the patient a therapeutically effective amount of a composition comprising 3-[(3′-fluoro-4-fluorobiphenyl-3-carbonyl) amino] phenoxyacetic acid isopropyl ester. In some embodiments, the 3-[(3′-fluoro-4-fluorobiphenyl-3-carbonyl) amino] phenoxyacetic acid isopropyl ester is administered to the periorbital skin of the patient as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

Provided herein is a method of treating glaucoma in a patient suffering from glaucoma comprising administering to the periorbital skin of the patient a therapeutically effective amount of a composition comprising 3-[(3′-fluoro-4-fluorobiphenyl-3-carbonyl) amino] phenoxyacetic acid isopropyl ester. In some embodiments, the 3-[(3′-fluoro-4-fluorobiphenyl-3-carbonyl) amino] phenoxyacetic acid isopropyl ester is administered to the periorbital skin of the patient as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

Provided herein is a method of lowering intraocular pressure in a patient suffering from glaucoma comprising administering to the periorbital skin of the patient a therapeutically effective amount of a composition comprising 3-[(3′-fluoro-4-fluorobiphenyl-3-carbonyl)amino]phenoxyacetic acid isopropyl ester. In some embodiments, the 3-[(3′-fluoro-4- fluorobiphenyl-3-carbonyl) amino] phenoxyacetic acid isopropyl ester is administered to the periorbital skin of the patient as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

In some embodiments, the 3-[(3′-fluoro-4-fluorobiphenyl-3-carbonyl)amino] phenoxyacetic acid isopropyl ester is administered to the periorbital skin of the patient as a topical composition to deliver to each eye from about 0.0005 micrograms to about 5.0 micrograms, about 0.001 micrograms to about 5.0 micrograms, about 0.002 micrograms to about 5.0 micrograms, about 0.005 micrograms to about 5.0 micrograms, 0.005 micrograms to about 2.0 micrograms, 0.005 micrograms to about 1.5 micrograms, about 0.01 micrograms to about 5.0 micrograms, about 0.05 micrograms to about 5.0 micrograms, about 0.075 micrograms to about 5.0 micrograms, about 0.1 micrograms to about 5.0 micrograms, about 0.01 micrograms to about 3.0 micrograms, about 0.05 micrograms to about 3.0 micrograms, about 0.075 micrograms to about 3.0 micrograms, about 0.1 micrograms to about 3.0 micrograms, about 0.01 micrograms to about 2.0 micrograms, about 0.05 micrograms to about 2.0 micrograms, about 0.075 micrograms to about 2.0 micrograms, about 0.1 micrograms to about 2.0 micrograms, about 0.01 micrograms to about 1.0 micrograms, about 0.05 micrograms to about 1.0 micrograms, about 0.075 micrograms to about 1.0 micrograms, about 0.1 micrograms to about 1.0 micrograms, about 0.0005 micrograms to about 0.5 micrograms, about 0.0005 micrograms to about 0.05 micrograms, about 0.0005 micrograms to about 0.005 micrograms, about 0.005 micrograms to about 0.5 micrograms, about 0.005 micrograms to about 0.05 micrograms, or about 0.05 micrograms to about 0.5 micrograms of 3-[(3′-fluoro-4-fluorobiphenyl-3-carbonyl) amino] phenoxyacetic acid isopropyl ester per administration. In some embodiments, the 3-[(3′-fluoro-4-fluorobiphenyl-3-carbonyl)amino] phenoxyacetic acid isopropyl ester is administered as a topical composition to the periorbital skin of the patient to deliver about 0.0005 micrograms, about 0.001 micrograms, about 0.0025 micrograms, about 0.005 micrograms, about 0.0075 micrograms, about 0.01 micrograms, about 0.025 micrograms, about 0.05 micrograms, about 0.07 micrograms, about 0.08 micrograms, about 0.09 micrograms, about 0.1 micrograms, about 0.2 micrograms, about 0.3 micrograms, about 0.4 micrograms, about 0.5 micrograms, about 0.6 micrograms, about 0.7 micrograms, about 0.8 micrograms, about 0.9 micrograms, about 1.0 micrograms, about 1.1 micrograms, about 1.2 micrograms, about 1.3 micrograms, about 1.4 micrograms, about 1.5 micrograms, about 1.75 micrograms, about 2.0 micrograms, about 2.25 micrograms, about 2.5 micrograms, about 3.0 micrograms, about 3.5 micrograms, about 4.0 micrograms, about 4.5 micrograms, or about 5.0 micrograms of 3-[(3′-fluoro-4-fluorobiphenyl-3-carbonyl) amino] phenoxyacetic acid isopropyl ester per administration.

In some embodiments, the 3-[(3′-fluoro-4-fluorobiphenyl-3-carbonyl)amino] phenoxyacetic acid isopropyl ester is administered to the periorbital skin of the patient as a topical composition to deliver to each eye about 1 microgram to about 2 micrograms. In some embodiments, the 3-[(3′-fluoro-4-fluorobiphenyl-3-carbonyl)amino] phenoxyacetic acid isopropyl ester is administered to the periorbital skin of the patient as a topical composition to deliver to each eye about 1 microgram to about 1.1 micrograms, about 1 microgram to about 1.2 micrograms, about 1 microgram to about 1.3 micrograms, about 1 microgram to about 1.4 micrograms, about 1 microgram to about 1.5 micrograms, about 1 microgram to about 1.6 micrograms, about 1 microgram to about 1.7 micrograms, about 1 microgram to about 1.8 micrograms, about 1 microgram to about 1.9 micrograms, about 1 microgram to about 2 micrograms, about 1.1 micrograms to about 1.2 micrograms, about 1.1 micrograms to about 1.3 micrograms, about 1.1 micrograms to about 1.4 micrograms, about 1.1 micrograms to about 1.5 micrograms, about 1.1 micrograms to about 1.6 micrograms, about 1.1 micrograms to about 1.7 micrograms, about 1.1 micrograms to about 1.8 micrograms, about 1.1 micrograms to about 1.9 micrograms, about 1.1 micrograms to about 2 micrograms, about 1.2 micrograms to about 1.3 micrograms, about 1.2 micrograms to about 1.4 micrograms, about 1.2 micrograms to about 1.5 micrograms, about 1.2 micrograms to about 1.6 micrograms, about 1.2 micrograms to about 1.7 micrograms, about 1.2 micrograms to about 1.8 micrograms, about 1.2 micrograms to about 1.9 micrograms, about 1.2 micrograms to about 2 micrograms, about 1.3 micrograms to about 1.4 micrograms, about 1.3 micrograms to about 1.5 micrograms, about 1.3 micrograms to about 1.6 micrograms, about 1.3 micrograms to about 1.7 micrograms, about 1.3 micrograms to about 1.8 micrograms, about 1.3 micrograms to about 1.9 micrograms, about 1.3 micrograms to about 2 micrograms, about 1.4 micrograms to about 1.5 micrograms, about 1.4 microgram s to about 1.6 micrograms, about 1.4 micrograms to about 1.7 micrograms, about 1.4 micrograms to about 1.8 micrograms, about 1.4 micrograms to about 1.9 micrograms, about 1.4 micrograms to about 2 micrograms, about 1.5 micrograms to about 1.6 micrograms, about 1.5 micrograms to about 1.7 micrograms, about 1.5 micrograms to about 1.8 micrograms, about 1.5 micrograms to about 1.9 micrograms, about 1.5 micrograms to about 2 micrograms, about 1.6 micrograms to about 1.7 micrograms, about 1.6 micrograms to about 1.8 micrograms, about 1.6 micrograms to about 1.9 micrograms, about 1.6 micrograms to about 2 micrograms, about 1.7 micrograms to about 1.8 micrograms, about 1.7 micrograms to about 1.9 micrograms, about 1.7 micrograms to about 2 micrograms, about 1.8 micrograms to about 1.9 micrograms, about 1.8 micrograms to about 2 micrograms, or about 1.9 micrograms to about 2 micrograms. In some embodiments, the 3-[(3′-fluoro-4-fluorobiphenyl-3-carbonyl)amino] phenoxyacetic acid isopropyl ester is administered to the periorbital skin of the patient as a topical composition to deliver to each eye about 1 microgram, about 1.1 micrograms, about 1.2 micrograms, about 1.3 micrograms, about 1.4 micrograms, about 1.5 micrograms, about 1.6 micrograms, about 1.7 micrograms, about 1.8 micrograms, about 1.9 micrograms, or about 2 micrograms. In some embodiments, the 3-[(3′-fluoro-4-fluorobiphenyl-3-carbonyl)amino] phenoxyacetic acid isopropyl ester is administered to the periorbital skin of the patient as a topical composition to deliver to each eye at least about 1 microgram, about 1.1 micrograms, about 1.2 micrograms, about 1.3 micrograms, about 1.4 micrograms, about 1.5 micrograms, about 1.6 micrograms, about 1.7 micrograms, about 1.8 micrograms, or about 1.9 micrograms. In some embodiments, the 3-[(3′-fluoro-4-fluorobiphenyl-3-carbonyl)amino] phenoxyacetic acid isopropyl ester is administered to the periorbital skin of the patient as a topical composition to deliver to each eye at most about 1.1 micrograms, about 1.2 micrograms, about 1.3 micrograms, about 1.4 micrograms, about 1.5 micrograms, about 1.6 micrograms, about 1.7 micrograms, about 1.8 micrograms, about 1.9 micrograms, or about 2 micrograms.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 0.1 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 0.2 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 0.3 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 0.4 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 0.5 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 0.6 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 0.7 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 0.8 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 0.9 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 1.0 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 1.1 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 1.2 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 1.3 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 1.4 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 1.5 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 1.6 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 1.7 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 1.8 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 1.9 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 2.0 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 2.5 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 3.0 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 3.5 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 4.0 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 4.5 micrograms per eye.

In some embodiments, the 3-[(3′-fluoro-4-fluorobiphenyl-3-carbonyl) amino] phenoxyacetic acid isopropyl ester is administered to the periorbital skin of the patient as a topical composition to deliver about 5.0 micrograms per eye.

In some embodiments, from about 10 micrograms per eye to about 30 micrograms per eye of the composition is administered to the periorbital skin of the patient. In some embodiments, about 3 micrograms per eye, about 5 micrograms per eye, about 10 micrograms per eye, about 15 micrograms per eye, about 20 micrograms per eye, about 25 micrograms per eye, or about 30 micrograms per eye of the composition is administered to the periorbital skin of the patient.

In some embodiments, the amount of ointment delivered to the periorbital skin of the patient is about 5 milligrams to about 30 milligrams. In some embodiments, the amount of ointment delivered to the periorbital skin of the patient is about 5 milligrams to about 8 milligrams, about 5 milligrams to about 10 milligrams, about 5 milligrams to about 12 milligrams, about 5 milligrams to about 15 milligrams, about 5 milligrams to about 17 milligrams, about 5 milligrams to about 20 milligrams, about 5 milligrams to about 25 milligrams, about 5 milligrams to about 30 milligrams, about 8 milligrams to about 10 milligrams, about 8 milligrams to about 12 milligrams, about 8 milligrams to about 15 milligrams, about 8 milligrams to about 17 milligrams, about 8 milligrams to about 20 milligrams, about 8 milligrams to about 25 milligrams, about 8 milligrams to about 30 milligrams, about 10 milligrams to about 12 milligrams, about 10 milligrams to about 15 milligrams, about 10 milligrams to about 17 milligrams, about 10 milligrams to about 20 milligrams, about 10 milligrams to about 25 milligrams, about 10 milligrams to about 30 milligrams, about 12 milligrams to about 15 milligrams, about 12 milligrams to about 17 milligrams, about 12 milligrams to about 20 milligrams, about 12 milligrams to about 25 milligrams, about 12 milligrams to about 30 milligrams, about 15 milligrams to about 17 milligrams, about 15 milligrams to about 20 milligrams, about 15 milligrams to about 25 milligrams, about 15 milligrams to about 30 milligrams, about 17 milligrams to about 20 milligrams, about 17 milligrams to about 25 milligrams, about 17 milligrams to about 30 milligrams, about 20 milligrams to about 25 milligrams, about 20 milligrams to about 30 milligrams, or about 25 milligrams to about 30 milligrams. In some embodiments, the amount of ointment delivered to the periorbital skin of the patient is about 5 milligrams, about 8 milligrams, about 10 milligrams, about 12 milligrams, about 15 milligrams, about 17 milligrams, about 20 milligrams, about 25 milligrams, or about 30 milligrams. In some embodiments, the amount of ointment delivered to the periorbital skin of the patient is at least about 5 milligrams, about 8 milligrams, about 10 milligrams, about 12 milligrams, about 15 milligrams, about 17 milligrams, about 20 milligrams, or about 25 milligrams. In some embodiments, the amount of ointment delivered to the periorbital skin of the patient is at most about 8 milligrams, about 10 milligrams, about 12 milligrams, about 15 milligrams, about 17 milligrams, about 20 milligrams, about 25 milligrams, or about 30 milligrams.

In some embodiments, the 3-[(3′-fluoro-4-fluorobiphenyl-3-carbonyl) amino] phenoxyacetic acid isopropyl ester is administered to the periorbital skin of each eye of the patient twice per day, once per day, once every other day, once every three days, once every four days, or once every seven days, or any combination thereof (e.g. a variable dosing protocol). In some embodiments, the 3-[(3′-fluoro-4-fluorobiphenyl-3-carbonyl) amino] phenoxyacetic acid isopropyl ester is administered to the periorbital skin of each eye of the patient once per week, twice per week, three times per week, once every two weeks, or once every three weeks. In some embodiments, the 3-[(3′-fluoro-4-fluorobiphenyl-3-carbonyl) amino] phenoxyacetic acid isopropyl ester is administered to the periorbital skin of each eye of the patient once per day.

In some embodiments, the 3-[(3′-fluoro-4-fluorobiphenyl-3-carbonyl) amino] phenoxyacetic acid isopropyl ester is administered to the periorbital skin of one eye of the patient twice per day, once per day, once every other day, once every three days, once every four days, or once every seven days, or any combination thereof (e.g. a variable dosing protocol). In some embodiments, the 3-[(3′-fluoro-4-fluorobiphenyl-3-carbonyl) amino] phenoxyacetic acid isopropyl ester is administered to the periorbital skin of one eye of the patient once per week, twice per week, three times per week, once a week, once every two weeks, or once every three weeks. In some embodiments, the 3-[(3′-fluoro-4-fluorobiphenyl-3-carbonyl) amino] phenoxyacetic acid isopropyl ester is administered to the periorbital skin of one eye of the patient once per day.

In some embodiments, the composition is applied to the periorbital skin using a device. In some embodiments, the device releases a preselected dosage in a uniform manner onto the periorbital skin of the patient. In some embodiments, the composition is applied by a roller device or a cotton swab to the periorbital skin. In some embodiments, the composition is applied by a roller device to the periorbital skin. In some embodiments, the composition is applied by a cotton swab to the periorbital skin. In some embodiments, the composition is applied by a spatula to the periorbital skin.

In some embodiments, the composition is administered to the periorbital skin above the upper eyelid, below the lower eyelid, or both above the upper and below the lower eyelids. In some embodiments, the composition is administered above the upper eyelid. In some embodiments, the composition is administered below the lower eyelid. In some embodiments, the composition is administered both above the upper and below the lower eyelid.

In some embodiments, the composition is effective to reduce the intraocular pressure of the patient. In some embodiments, the composition is effective to reduce the intraocular pressure of the patient by at least 1 mmHg, by at least 2 mmHg, by at least 3 mmHg, by at least 4 mmHg, by at least 5 mmHg, by at least 6 mmHg, by at least 7 mmHg, by at least 8 mmHg, by at least 9 mmHg, by at least 10 mmHg, by at least 11 mmHg, by at least 12 mmHg, by at least 13 mmHg, by at least 14, by at least 15 mmHg, by at least 16 mmHg, by at least 17 mmHg, or by at least 18 mmHg. In some embodiments, the reduced intraocular pressure is measured after a single administration of the composition. In some embodiments, the reduced intraocular pressure is measured after two, three, four, five, six, or seven once-daily administrations of the composition.

### Certain Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and sub combinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, may “consist of” or “consist essentially of” the described features.

“Treating” or “treatment” as used herein includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e ., not worsening) the state of disease, delay or slowing of disease progression, amelioration, diminishment of the reoccurrence of disease. Treatment may prevent the disease from occurring; relieve the disease's symptoms, fully or partially remove the disease's underlying cause, shorten a disease's duration, or do a combination of the above.

“Treating” and “treatment” as used herein may also include prophylactic treatment. Treatment methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for duration sufficient to treat the patient.

The term “periorbital” refers to the area surrounding the socket or orbit of the eye (see Mosby's medical dictionary).

The term “preorbital” refers to the area in front of the orbit or eye socket.

The term “eyelid” refers to movable folds of the skin over the eye (see Mosby's medical dictionary).

“OD” refers to the right eye.

“OS” refers to the left eye.

“OU” refers to both eyes.

“Periorbital administration” involves administration to the periorbital skin and specifically excludes administration to the upper eyelid, lower eyelid, and eyelid margin.

The term “elevated intraocular pressure” refers to an intraocular pressure above that of normal intraocular pressure. In some embodiments, elevated intraocular pressure is an intraocular pressure of above about 21 mmHg. In some embodiments, elevated intraocular pressure is an intraocular pressure of above about 20 mmHg, above ab out 21 mmHg, above about 22 mmHg, above about 23 mmHg, or above about 24 mmHg.

The term “normal intraocular pressure” refers to the intraocular pressure in a healthy individual. In some embodiments, normal intraocular pressure is about 20 mmHg, about 19 mmHg, about 18 mmHg, about 17 mmHg, about 16 mmHg, about 15 mmHg, about 14 mmHg, about 13 mmHg, about 12 mmHg, about 11 mmHg, or about 10 mmHg. In some embodiments, normal intraocular pressure is from about 10 mmHg to about 21 mmHg, from about 10 mmHg to about 20 mmHg, or from about 12 mmHg to about 20 mmHg.

The term “lotion” describes an emulsion liquid dosage form. This dosage form is generally for external application to the skin (US FDA Drug Nomenclature Monograph, number C-DRG-00201).

The term “cream” describes an emulsion semisolid dosage form, usually containing >20% water and volatiles and/or <50% hydrocarbons, waxes or polyols as the vehicle. A cream is more viscous than a lotion. This dosage form is generally for external application to the skin (US FDA Drug Nomenclature Monograph, number C-DRG-00201).

The term “ointment” describes a semisolid dosage form, usually containing <20% water and volatiles and/or >50% hydrocarbons, waxes or polyols as the vehicle. This dosage form is generally for external application to the skin or mucous membranes (US FDA Drug Nomenclature Monograph, number C-DRG-00201).

The term “solution” describes a clear, homogeneous liquid dosage form that contains one or more chemical substances dissolved in a solvent or mixture of mutually miscible solvents (US FDA Drug Nomenclature Monograph, number C-DRG-00201).

The term “suspension” refers to a heterogeneous mixture containing solid particles that are sufficiently large for sedimentation.

The term “serum” refers to a clear gel-based or liquid formulation designed to deliver high concentrations of specific active ingredients to the skin.

### EXAMPLES

### Example 1: Effect of JV-GL1 on Intraocular Pressure (MP) Administered as a Single Periorbital Dose

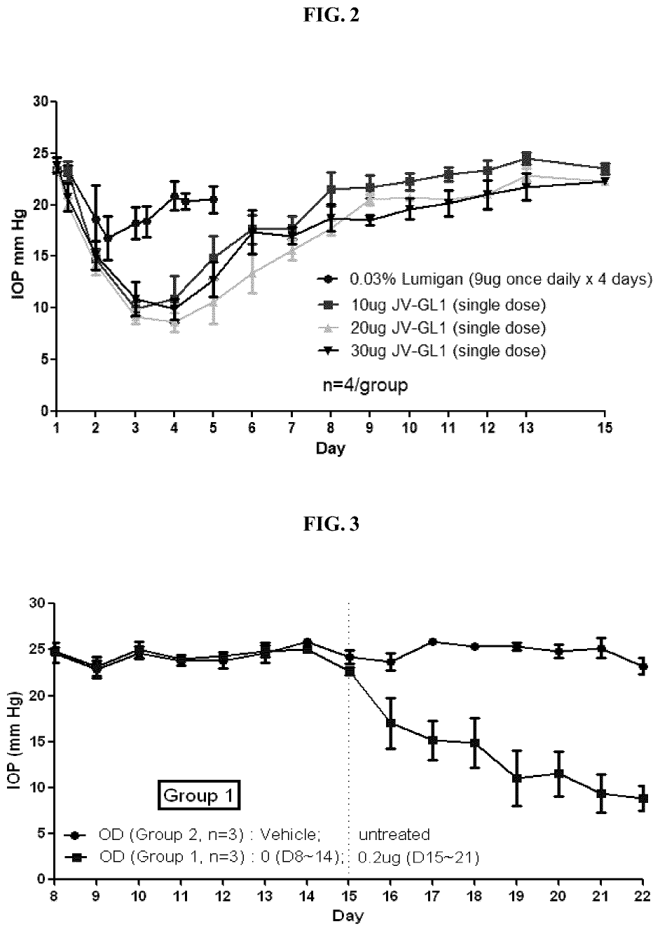

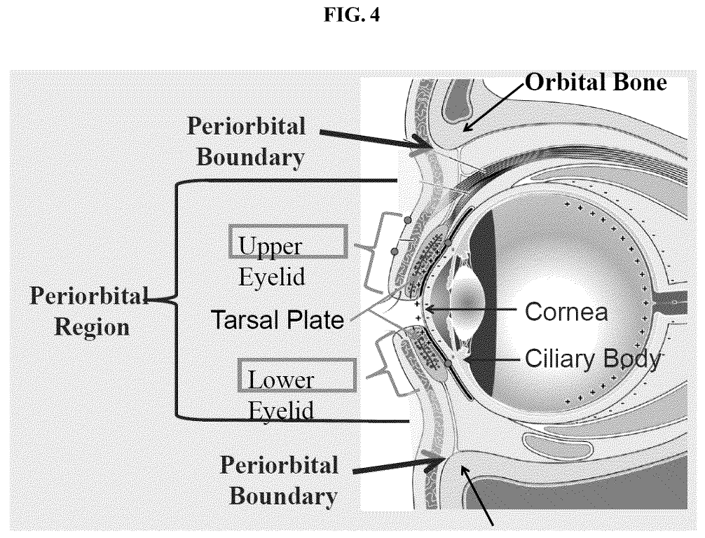

Ocular normotensive cynomolgus monkeys were administered a single dose of JV-GL1 Ointment (20% medium-chain triglycerides and 80% petrolatum (v/v)) to the periorbital skin of the right eye (OD) at JV-GL1 doses of 10 micrograms, 20 micrograms, and 30 micrograms and TOP was measured; Lumigan® eye drops were used as a comparator. JV-GL1 Ointment administered as a single periorbital dose had a significant and long-acting TOP lowering effect in ocular normotensive cynomolgus monkeys (FIG. 2). Compared with baseline, IOP was decreased in the eyes (OD) of animals treated with 10 micrograms, 20 micrograms, and 30 micrograms JVGL-1 to a similar extent (approximately 60%) whereas a much lower decrease occurred for Lumigan® (approximately 30%).

Periorbital skin redness, swelling and pupil constriction were observed in the OD of the animals in three JV-GL1 treated groups at 2-3 hours after dosing. The incidence rate of periorbital skin redness in all three test article groups was 100% at 2-3 hours post-dosing, and completely recovered by Day 2. The periorbital skin swelling disappeared by Day 5, and the incidence rate showed a certain dose dependent relationship during the study. The incidence rate of pupil constriction in the three test article groups was 100% after 2-3 hours post-dosing. Except for a single animal in the high-dose group, the pupil constriction in other animals recovered by Day 15.

### Example 2: IOP after Periorbital Administration of 0.2 μg/day/eye as an Ointment

Ocular normotensive cynomolgus monkeys (Group 1, n=3) were unilaterally treated via periorbital skin administration of JV-GL1 ointment once daily in an amount of 0.2 μg in the right eye on Days 15-21 of a study, the results of which are shown in FIG. 3. JV-GL1 was administered as an ointment (20% medium-chain triglycerides and 80% petrolatum (v/v)). The animals were untreated on days 8-14 and IOP monitored daily. Group 2 (n=3) was administered vehicle ointment alone on days 8-14, followed by no treatment on days 15-21 of the study.

As shown in the Group 2 data of FIG. 3, animals treated with vehicle alone showed no change from an untreated state. Conversely, upon daily administration of 0.2 μg/eye (OD) over days 15-21 in Group 1, the animals displayed a marked decrease in IOP as early as one day following initiation of treatment, dropping from a starting point of ˜25 mmHg and reaching ˜10 mmHg by the end of the study. These results suggest that surprisingly low doses of JV-GL1 can be effective for reduction of intraocular pressure when administered to the periorbital skin as an ointment.

Separately, ocular normotensive cynomolgus monkeys were administered single doses of the ointment at 0.6 micrograms, 2.0 micrograms or 5.0 micrograms per eye. The skin redness and swelling disappeared a few hours to overnight after dosing. During the dosing period, persistent pupil constriction was observed in the eyes treated with either eye drops or ointment. This data suggests that low doses of JV-GL1 may be preferable to avoid side effects seen at higher single doses, particularly for daily administration.

### Example 3: Toxicology Study of JV-GL1 Administered to the Periorbital Skin in Cynomolgus Monkeys

In a comprehensive, GLP, 4-week, repeat-dose toxicology study in cynomolgus monkeys (n =6/sex/group), JV-GL1 Ointment (0.058, 0.145 and 0.290 mg/g) was administered via periorbital application to both eyes at respective JV-GL1 doses of 1, 2.5 and 5 micrograms/eye/day (2, 5, 10 micrograms/day). Groups of 4 monkeys/sex each were terminated on Day 29 and recovery animals (n=2/sex/group) were terminated on Day 43 after a 14 -day recovery

All animals survived to their scheduled necropsies and the ointments were well tolerated.

Ophthalmoscopic examinations using a slit lamp and corneal fluorescein staining revealed no abnormal findings in the anterior segment (eyelid, cornea, sclera, conjunctiva, iris and lens) or fundus of any of the monkeys (control or JV-GL1 Ointment treated), and no adverse effects on ocular function.

The only in-life findings were periorbital skin redness (site of application) and pupil constriction. Both increased in incidence and/or severity with increasing ointment concentration. The local irritation began on Day 1, resolved prior to the next day's dose and decreased in incidence within 5 to 6 days of treatment initiation. The pupil constriction also began early in the study (Days 1 to 3) and did not resolve prior to the next day's dose. During the recovery period, these findings resolved.

The NOAEL (no-observed-adverse-effect level) was 5 micrograms/eye/day, the highest dose tested.

Periorbital skin administration of JV-GL1 proved to be as effective as eye drops in a monkey study using essentially equal doses. In the initial periorbital dosing pharmacodynamic study, doses of JV-GL1 (in ointment) ranging from 10 to 30 micrograms/eye resulted in modest pupil constriction, erythema of the ocular surface and periorbital skin, and swelling of the periorbital skin. When lower doses of 0.2 to 5 micrograms/eye were administered, the efficacious effects ofJV-GL1 were retained without obvious periorbital skin swelling and hyperemia of the periorbital skin and ocular surface in monkeys.

### Example 4: A Human Safety and Efficacy Study of JV-GL1 Applied to the Periorbital Skin in Subjects with Open-Angle Glaucoma or Ocular Hypertension

A study to assess the safety, tolerability and feasibility of lowering intraocular pressure using an experimental study ointment containing JV-GL1 is performed. The study includes two parts: Part 1 assesses safety and tolerability, and Part 2 assesses safety and effectiveness of JV-GL1. A description of the study is shown below in Table 1.

TABLE 1
Description of study design to measure the safety and efficacy of
JV-GL1 ointment in humans

| Condition or | |
|---|---|
| disease | Intervention/treatment |
| Open Angle | Drug: |
| Glaucoma | Part 1 and Part 2: JV-GL1 topical ointment dose |
| Ocular | escalation |
| Hypertension | Part 2 Active Control: Topical Latanoprost |
| Study Type: | Interventional (Clinical Trial) |
| Estimated | Part 1: 18 participants |
| Enrollment: | Part 2: 50 participants |
| Intervention | This is a single-center study to assess the safety and |
| Model | feasibility of JV-GL1 as a treatment for open angle |
| Description: | glaucoma or ocular hypertension. The study has two |
| | parts: Part 1 is an open-label dose escalation study |
| | with up to 18 participants. Six subjects in each of the |
| | 5 cohorts (Dose levels 1~5) receive JV-GL1 for 9 |
| | days. Up to 2 doses of JV-GL1 from Part 1 are |
| | selected to proceed to Part 2 of the study. Up to 50 |
| | evaluable subjects are randomized to receive either |
| | JV-GL1 at one of the 2 selected doses, or 0.005% |
| | latanoprost eye drops for 28 days. |

This is a first-in-human, open-label, Phase 1/2 study in adult subjects with open-angle glaucoma or ocular hypertension. The study is divided into 2 parts.

Part 1 is dose escalation where up to 5 doses are tested. Subjects in Part 1 are dosed once daily for 9 days and exited on Day 15.

Part 2 of the study is a randomized Experimental Dose A: Experimental Dose B: Active Control, treating additional subjects at the selected JV-GL1 doses or Latanoprost for 28 days.

10 subjects enrolled in Part 2 JV-GL1 arm have pharmacokinetic assessments performed on Day 1 and Day 28.

The dose escalation study is divided into various arms, each with different interventions as shown below in Table 2.

TABLE 2
Arms of the study to measure the safety and efficacy of periorbital
administration of JV-GL1 ointment

| Arm | Intervention/treatment |
|---|---|
| Experimental: Dose Level 1 JV-GL1 Ointment | Drug: JV-GL1 Ointment |
| Experimental: Dose Level 2 JV-GL1 Ointment | Drug: JV-GL1 Ointment |
| Experimental: Dose Level 3 JV-GL1 Ointment | Drug: JV-GL1 Ointment |
| Experimental: Dose Level 4 JV-GL1 Ointment | Drug: JV-GL1 Ointment |
| Experimental: Dose Level 5 JV-GL1 Ointment | Drug: JV-GL1 Ointment |

The primary outcomes of the study are safety and tolerability (including incidence of treatment emergent Adverse Events) and intraocular pressure (IOP) change from baseline (mmHg) measured with a Goldman applanation tonometer.

Secondary outcome measurements of the study include TOP reduction by JV-GL1 versus latanoprost.

The study is available to individuals of both sexes aged 18 years and older. Healthy volunteers are not accepted. Eligible individuals are adults at least 18 years of age willing and able to give informed consent and in the investigator's judgment able to follow the study protocol. Eligible individuals must also have a d diagnosis of bilateral open-angle glaucoma or ocular hypertension.

### Example 5: Application of an Ointment Comprising JV-GL1

The JV-GL1 ointment tube should is stored at room temperature away from direct sunlight. A subject prescribed the ointment is instructed to use the medication once per day for 28 days at about the same time every day.

Each administration of the ointment is performed as follows:

The subject ensures the skin around the eyes is free of makeup, clean and dry. The patient ensures he or she has all of the materials needed for the administration at hand (a sterile cotton swab, the tube with the ointment, clean tissue or paper towel to clean swab while measuring the ointment, and a mirror). The subject then notes the date and time they are administering the ointment. The subject washes and dries his or her hands.

The subject removes one cotton swab from an available packet and places it on the tissue or paper towel. The tube containing the ointment is then unscrewed. 1.5 cm of the ointment is measured out from the tube by squeezing the tube with one hand and measuring with a ruler. The 1.5 cm of ointment is then placed on the tip of the cotton swab.

The tip of the cotton swab is then applied to the top of the right eyelid of the subject next to the nose. The ointment is then evenly spread to the upper and lower periorbital skin region of the eye. The subject can monitor this process in front of the mirror to ensure even spread. The cotton swab is then thrown away and the process repeated for the left eye.

The lid is then screwed back on the ointment tube. The subject then washes his or her hands. The subject does not rub, wash, or apply any makeup, lotion, or other product to the skin around the eyes for two hours after application.

### Example 6: IOP in Human Subjects after Periorbital Administration of 0.3 μg/day/eye—4 μg/day/eye of JV-GL1

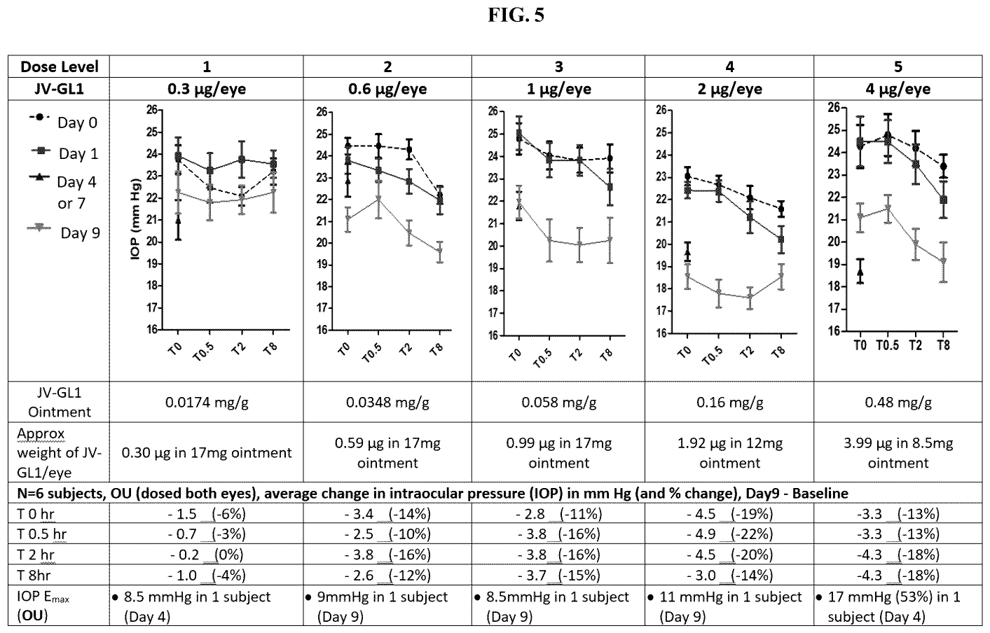

Human subjects were treated with JV-GL1 ointment (20% medium-chain triglycerides and 80% petrolatum (v/v)) via periorbital skin administration to both eyes once daily. The human subjects were monitored daily for 9 days, with TOP measurements taken at 0 hours, 0.5 hours, 2 hours, and 8 hours from administration of the dose on Days 0, 1, and 9. IOP measurements were taken 0 hours from administration of the dose on Days 4 and 7. Results are shown in FIG. 5.

Subjects in Group 1 (n=6) were treated with 0.30 μg/eye/day of JV-GL1 in 17 mg ointment (0.0018% w/w). Upon daily administration of 0.30 μg/eye over 9 days, the average change in TOP at 0 hours, 0.5 hours, 2 hours, and 8 hours after dose administration was −6%, −3%, 0%, and −4%, respectively.

Subjects in Group 2 (n=6) were treated with 0.59 μg/eye/day of JV-GL1 in 17 mg ointment (0.0035% w/w). Upon daily administration of 0.59 μg/eye over 9 days, the average change in TOP at 0 hours, 0.5 hours, 2 hours, and 8 hours after dose administration was −14%, −10%, −16%, and −12%, respectively.

Subjects in Group 3 (n=6) were treated with 0.99 μg/eye/day of JV-GL1 in 17 mg ointment (0.0058% w/w). Upon daily administration of 0.99 μg/eye over 9 days, the average change in TOP at 0 hours, 0.5 hours, 2 hours, and 8 hours after dose administration was −11%, −16%, −16%, and −15%, respectively.

Subjects in Group 4 (n=6) were treated with 1.92 μg/eye/day of JV-GL1 in 12 mg ointment (0.0160% w/w). Upon daily administration of 1.92 μg/eye over 9 days, the average change in TOP at 0 hours, 0.5 hours, 2 hours, and 8 hours after dose administration was −19%, −22%, −20%, and −14%, respectively.

Subjects in Group 5 (n=6) were treated with 3.99 μg/eye/day of JV-GL1 in 8.5 mg ointment (0.0469% w/w). Upon daily administration of 3.99 μg/eye over 9 days, the average change in TOP at 0 hours, 0.5 hours, 2 hours, and 8 hours after dose administration was −13%, −13%, −18%, and −18%, respectively.

As shown in FIG. 5, there is a dose-dependent trend in TOP reduction with Groups 1, 2, 3, and 4. During the dosing period, subjects in Groups 1-4 did not report any irritation or discomfort associated with AE of mild periorbital erythema. These results suggest that surprisingly low doses of JV-GL1 can be effective for reduction of intraocular pressure when administered to the periorbital skin as an ointment.

What is claimed is:

1. A method of lowering intraocular pressure in a patient suffering from elevated intraocular pressure comprising administering a therapeutically effective amount of a composition comprising 3-[(3′-fluoro-4-fluorobiphenyl-3-carbonyl) amino] phenoxyacetic acid isopropyl ester to the periorbital skin of an eye of the patient wherein the 3-[(3′-fluoro-4-fluorobiphenyl-3-carbonyl) amino] phenoxyacetic acid isopropyl ester is administered as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

2. A method of treating glaucoma in a patient suffering from glaucoma comprising administering a therapeutically effective amount of a composition comprising 3-[(3′-fluoro-4-fluorobiphenyl-3-carbonyl) amino] phenoxyacetic acid isopropyl ester to the periorbital skin of an eye of the patient wherein the 3-[(3′-fluoro-4-fluorobiphenyl-3-carbonyl) amino] phenoxyacetic acid isopropyl ester is administered as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

3. A method of lowering intraocular pressure in a patient suffering from glaucoma comprising administering a therapeutically effective amount of a composition comprising 3-[(3′-fluoro-4-fluorobiphenyl-3-carbonyl)amino]phenoxyacetic acid isopropyl ester to the periorbital skin of an eye of the patient wherein the 3-[(3′-fluoro-4-fluorobiphenyl-3-carbonyl)amino] phenoxyacetic acid isopropyl ester is administered as a topical composition to deliver from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

4. The method of claim 1, 2, or 3, wherein the composition comprising 3-[(3′-fluoro-4-fluorobiphenyl-3-carbonyl) amino] phenoxyacetic acid isopropyl ester is administered to the periorbital skin of each eye of the patient twice per day, once per day, once every other day, once every three days, once every four days, or once every seven days.

5. The method of claim 4, wherein the topical composition is administered once per day.

6. The method of any one of claims 1-5, wherein the composition is in the form of an ointment.

7. The method of claim 6, wherein the ointment comprises petrolatum.

8. The method of claim 6, wherein the ointment comprises petrolatum and medium-chain triglycerides.

9. The method of claim 6, wherein the medium-chain triglycerides comprise a mixture of C6, C8, C10 and C12 fatty acids.

10. The method of claim 9, wherein the medium-chain triglycerides comprise a mixture of caprylic acid and capric acid.

11. The method of any one of claims 6-10, wherein the ointment comprises petrolatum and medium-chain triglyceride in the ratio of about 1:1 (v/v), about 2:1 (v/v), about 3:1 (v/v), about 4:1 (v/v), about 5:1 (v/v), or about 6:1 (v/v).

12. The method of claim 11, wherein the ointment comprises petrolatum and medium-chain triglyceride in the ratio of about 4:1 (v/v).

13. The method of any one of claims 1-12, wherein the composition is administered from a device which releases a preselected dosage in a uniform manner onto the periorbital skin of the patient.

14. The method of any one of claims 1-13, wherein the method comprises administering the composition to the periorbital skin above the upper eyelid, below the lower eyelid, or both above the upper and below the lower eyelids.

15. The method of claim 14, wherein the method comprises administering the composition to the periorbital skin above the upper eyelid.

16. The method of claim 14, wherein the method comprises administering the composition to the periorbital skin below the lower eyelid.

17. The method of claim 14, wherein the method comprises administering the composition to the periorbital skin both above the upper and below the lower eyelids.

18. The method of any one of claims 1-17, wherein the composition is administered as a topical composition to deliver from about 0.0005 micrograms per eye of 3-[(3′-fluoro-4-fluorobiphenyl-3-carbonyl) amino] phenoxyacetic acid isopropyl ester per administration to about 0.005 micrograms per eye of 3-[(3′-fluoro-4-fluorobiphenyl-3-carbonyl) amino] phenoxyacetic acid isopropyl ester per administration.

19. The method of any one of claims 1-17, wherein the composition is administered as a topical composition to deliver from about 0.005 micrograms per eye of 3-[(3′-fluoro-4-fluorobiphenyl-3-carbonyl) amino] phenoxyacetic acid isopropyl ester per administration to about 0.05 micrograms per eye of 3-[(3′-fluoro-4-fluorobiphenyl-3-carbonyl) amino] phenoxyacetic acid isopropyl ester per administration.

20. The method of any one of claims 1-17, wherein the composition is administered as a topical composition to deliver from about 0.05 micrograms per eye of 3-[(3′-fluoro-4-fluorobiphenyl-3-carbonyl) amino] phenoxyacetic acid isopropyl ester per administration to about 0.5 micrograms per eye of 3-[(3′-fluoro-4-fluorobiphenyl-3-carbonyl) amino] phenoxyacetic acid isopropyl ester per administration.

21. The method of any one of claims 1-17, wherein the composition is administered as a topical composition to deliver from about 0.5 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

22. The method of any one of claims 1-17, wherein the composition is administered as a topical composition to deliver about 0.3 micrograms per eye of 3-[(3′-fluoro-4-fluorobiphenyl-3-carbonyl) amino] phenoxyacetic acid isopropyl ester per administration.

23. The method of any one of claims 1-17, wherein the composition is administered as a topical composition to deliver about 0.6 micrograms per eye of 3-[(3′-fluoro-4-fluorobiphenyl-3-carbonyl) amino] phenoxyacetic acid isopropyl ester per administration.

24. The method of any one of claims 1-17, wherein the composition is administered as a topical composition to deliver about 1.0 micrograms per eye of 3-[(3′-fluoro-4-fluorobiphenyl-3-carbonyl) amino] phenoxyacetic acid isopropyl ester per administration.

25. A pharmaceutical composition suitable for topical periorbital administration, comprising 3-[(3′-fluoro-4-fluorobiphenyl-3-carbonyl) amino] phenoxyacetic acid isopropyl ester and a pharmaceutically acceptable excipient, wherein the pharmaceutical composition is configured to dispense from about 0.0005 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

26. The pharmaceutical composition of claim 25, wherein the composition is in the form of an ointment.

27. The pharmaceutical composition of claim 26, wherein the ointment comprises petrolatum.

28. The pharmaceutical composition of claim 26, wherein the ointment comprises petrolatum and medium-chain triglycerides.

29. The pharmaceutical composition of claim 28, wherein the medium-chain triglyceride comprises a C6, a C8, a C10, or a C12 fatty acid, or any combination thereof.

30. The pharmaceutical composition of claim 29, wherein the medium-chain triglycerides comprise a mixture of caprylic acid and capric acid.

31. The pharmaceutical composition of any one of claims 28-30, wherein the ointment comprises petrolatum and medium chain triglycerides in the ratio of about 1:1 (v/v), about 2:1 (v/v), about 3:1 (v/v), about 4:1 (v/v), about 5:1 (v/v), or about 6:1 (v/v).

32. The pharmaceutical composition of claim 31, wherein the ointment comprises petrolatum and medium chain triglycerides in the ratio of about 4:1 (v/v).

33. The pharmaceutical composition of any one of claims 25-32, wherein the composition is administered from a device which releases a preselected dosage in a uniform manner onto the periorbital skin of the patient.

34. The pharmaceutical composition of any one of claims 25-33, wherein the composition is configured to deliver from about 0.0005 micrograms per eye of 3-[(3′-fluoro-4-fluorobiphenyl-3-carbonyl) amino] phenoxyacetic acid isopropyl ester per administration to about 0.005 micrograms per eye of 3-[(3′-fluoro-4-fluorobiphenyl-3-carbonyl) amino] phenoxyacetic acid isopropyl ester per administration.

35. The pharmaceutical composition of any one of claims 25-33, wherein the composition is configured to deliver from about 0.005 micrograms per eye of 3-[(3′-fluoro-4-fluorobiphenyl-3-carbonyl) amino] phenoxyacetic acid isopropyl ester per administration to about 0.05 micrograms per eye of 3-[(3′-fluoro-4-fluorobiphenyl-3-carbonyl) amino] phenoxyacetic acid isopropyl ester per administration.

36. The pharmaceutical composition of any one of claims 25-33, wherein the composition is configured to deliver from about 0.05 micrograms per eye of 3-[(3′-fluoro-4-fluorobiphenyl-3-carbonyl) amino] phenoxyacetic acid isopropyl ester per administration to about 0.5 micrograms per eye of 3-[(3′-fluoro-4-fluorobiphenyl-3-carbonyl) amino] phenoxyacetic acid isopropyl ester per administration.

37. The pharmaceutical composition of any one of claims 25-33, wherein the composition is configured to deliver from about 0.5 micrograms per eye of 3-[(3′-fluoro-4-fluorobiphenyl-3-carbonyl) amino] phenoxyacetic acid isopropyl ester per administration to about 5.0 micrograms per eye of 3-[(3′-fluoro-4-fluorobiphenyl-3-carbonyl) amino] phenoxyacetic acid isopropyl ester per administration.

38. The pharmaceutical composition of any one of claims 25-33, wherein the composition is configured to deliver about 0.3 micrograms per eye of 3-[(3′-fluoro-4-fluorobiphenyl-3-carbonyl) amino] phenoxyacetic acid isopropyl ester per administration.

39. The pharmaceutical composition of any one of claims 25-33, wherein the composition is configured to deliver about 0.6 micrograms per eye of 3-[(3′-fluoro-4-fluorobiphenyl-3-carbonyl) amino] phenoxyacetic acid isopropyl ester per administration.

40. The pharmaceutical composition of any one of claims 25-33, wherein the composition is configured to deliver about 1.0 micrograms per eye of 3-[(3′-fluoro-4-fluorobiphenyl-3-carbonyl) amino] phenoxyacetic acid isopropyl ester per administration.

41. The pharmaceutical composition of any one of claims 25-33, wherein the composition is configured to deliver about 2.0 micrograms per eye of 3-[(3′-fluoro-4-fluorobiphenyl-3-carbonyl) amino] phenoxyacetic acid isopropyl ester per administration.

42. The pharmaceutical composition of any one of claims 25-33, wherein the composition is configured to deliver about 3.0 micrograms per eye of 3-[(3′-fluoro-4-fluorobiphenyl-3-carbonyl) amino] phenoxyacetic acid isopropyl ester per administration.

43. The pharmaceutical composition of any one of claims 25-33, wherein the composition is configured to deliver about 4.0 micrograms per eye of 3-[(3′-fluoro-4-fluorobiphenyl-3-carbonyl) amino] phenoxyacetic acid isopropyl ester per administration.

44. The pharmaceutical composition of any one of claims 25-33, wherein the composition is configured to deliver about 5.0 micrograms per eye of 3-[(3′-fluoro-4-fluorobiphenyl-3-carbonyl) amino] phenoxyacetic acid isopropyl ester per administration.
